(19) United States
(12) Patent Application Publication
(10) Pub. No.: US 2021/0282856 A1
(43) Pub. Date: Sep. 16, 2021

(54) Smart Surgical Laser Tissue Sealing And Cutting Apparatus With Optical Fiber Guided Sensors

(71) Applicants: Jay Eunjae Kim, Belleve, WA (US); Gerald Ho Kim, Ontario, CA (US)

(72) Inventors: Jay Eunjae Kim, Belleve, WA (US); Gerald Ho Kim, Ontario, CA (US)

(21) Appl. No.: 17/334,795

(22) Filed: May 31, 2021

Related U.S. Application Data
(63) Continuation-in-part of application No. 16/505,680, filed on Jul. 8, 2019.
(60) Provisional application No. 62/696,857, filed on Jul. 12, 2018.

(51) Int. Cl. A61B 18/22

(52) U.S. Cl. CPC A61B 18/22; A61B 2018/00601

(57) ABSTRACT

Embodiments of an apparatus and method for sealing and cutting of tissue during surgeries, especially in general, endoscopic, laparoscopic and robotic, are described. In one aspect, an apparatus comprises a laser system and a laser beam delivery unit. The laser system comprises a tissue cutting laser configured to emit a first laser beam to cut a tissue. The laser system also comprises a tissue sealing laser configured to emit a second laser beam to seal the tissue. The laser beam delivery unit is detachably coupled to the laser system and is configured to guide and direct the first and second laser beams to cut and seal the tissue.

### CROSS REFERENCE TO RELATED PATENT APPLICATION(S)

The present disclosure is part of a continuation-in-part (CIP) of U.S. patent application Ser. No. 16/505,680, filed on 8 Jul. 2019, which is a non-provisional patent application claiming the priority benefit of U.S. Provisional Patent Application No. 62/696,857, filed on 12 Jul. 2018. Contents of aforementioned applications are herein incorporated by reference in their entirety.

### BACKGROUND

### Technical Field

The present disclosure generally relates to the field of medical device and, more particularly, to a tissue sealing and cutting device using laser to perform sealing and cutting of tissues, especially, in general, endoscopic, laparoscopic and robotic surgeries.

### Description of the Related Art

Presently, uncontrollable bleeding during surgery is addressed with electrocauterization devices such as monopolar and bipolar systems based on high electric currents. However, there is an intrinsic risk in the use of electrocauterization devices in an operating room due to exposure of high electric currents to patients and medical staff. As an electric current traverses through the body of a patient when an electrocauterization system is used, whether monopolar or bipolar, electric shock marks or burn marks on the patient (e.g., on the ear) tend to result. For example, monopolar systems can cause thermal injury to surrounding tissues and accidental burns if used incorrectly. One main issue with monopolar devices is the interference with automatic implantable cardioverter defibrillator (AICD) as the monopolar device may trigger accidental shock to the patient. To avoid accidental triggering, the AICD may need to be turned off prior to the use of a monopolar device on the patient. Additionally, the AICD may need to be re-programmed afterwards.

Moreover, the use of saline solution during surgery tends to be limited when a high electric current system is in use. Further, the use of electrocauterization systems may not be suitable for patients with metal implants.

### SUMMARY

Various embodiments disclosed herein pertain to an apparatus and method for sealing and cutting of tissues during surgeries, especially general, endoscopic, laparoscopic and robotic surgeries. The apparatus comprises a laser system and a laser beam delivery unit. The laser system emits tunable laser beam with a wavelength suitable for sealing and cutting of a soft tissue. The laser beam delivery unit is disposable and is used to guide the laser beam to where tissue sealing and cutting is needed. Areas surrounding the tissue where tissue sealing and cutting occur are cooled by a liquid dispensed from a probe of the laser beam delivery unit. Fiber guided sensors of optical feedback from a photodiode and a pyrometer (or non-contact temperature sensor) detect reflected energy and temperature of a tissue, respectively. Other fiber guided sensors provide a distance measurement and a target probing on a tissue. All these optical fiber and non-optical sensors provide the smart laser system to run at safer and optimum conditions for tissue sealing and cutting operations during surgeries.

The proposed techniques are further described below in the detailed description. This summary is not intended to identify essential features of the claimed subject matter, nor is it intended for use in determining the scope of the claimed subject matter.

### BRIEF DESCRIPTION OF THE DRAWINGS

The accompanying drawings are included to provide a further understanding of the disclosure and are incorporated in and constitute a part of the present disclosure. The drawings illustrate embodiments of the disclosure and, together with the description, serve to explain the principles of the disclosure. It is appreciable that the drawings are not necessarily in scale as some components may be shown to be out of proportion than the size in actual implementation in order to clearly illustrate the concept of the present disclosure.

FIG. 1 is a laser tissue sealing and cutting apparatus in accordance with an embodiment of the present disclosure.

FIG. 2 is a plan view of the laser tissue sealing and cutting apparatus of FIG. 1 with a detail view of emitting laser beams at the probe end of a laser beam delivery unit.

FIG. 3 is an enlarged view of the probe end of a laser beam delivery unit of the laser tissue sealing and cutting apparatus of FIG. 1.

FIG. 4 shows detachment of a laser beam delivery unit from a laser system of the laser tissue sealing and cutting apparatus of FIG. 1.

FIG. 5 shows a laser depth measurement device, which, sensing via optical fibers, is a sensor component of the laser tissue sealing and cutting apparatus of FIG. 1.

FIG. 6 shows non-contact temperature sensor such a pyrometer, which, sensing via optical fibers, is a sensor component of the laser tissue sealing and cutting apparatus of FIG. 1.

FIG. 7 is a laser tissue sealing and cutting apparatus with two laser sources in accordance with an embodiment of the present disclosure.

FIG. 8 shows detachment of a laser beam delivery unit from a laser system of the laser tissue sealing and cutting apparatus of FIG. 7.

FIG. 9 is a flowchart of a process of tissue sealing and cutting from a single laser source apparatus in accordance with the present disclosure.

FIG. 10 is a flowchart of a process of tissue sealing and cutting from a dual laser source apparatus in accordance with the present disclosure.

FIG. 11 is a diagram of an example apparatus in an implementation in accordance with the present disclosure.

The following is a listing of numerated components in all figures:
10 probe laser;
11 beam splitter;
12 beam splitter;
20 tissue sealing and cutting laser;
21 optical fiber;
22 tissue sealing laser;
23 optical fiber;
24 tissue cutting laser;
25 optical fiber;
30 air tank;
31 water tank;
36 tubing for air;
37 tubing for water;
40 pyrometer (example of non-contact temperature sensors);
41 depth sensor;
42 photodiode;
43 guide laser;
44 temperature sensor;
45 optical fiber of pyrometer;
46 optical fiber of depth sensor;
47 optical fiber of guide laser;
48 wire of temperature sensor;
50 laser coupling of tissue cutting beam;
51 laser coupling of tissue sealing beam;
55 motor;
56 switching plate;
58 optical fiber of laser cutting beam;
59 optical fiber of laser sealing beam;
61 fiber coupling of male;
62 coupling block of male of air and water;
63 fiber coupling of female;
64 coupling block of female of air and water;
65 optical fiber for reflected laser beam to photodiode 42;
66 air tube;
67 water tube;
70 fiber optic sheathing;
75 enclosure;
76 enclosure;
78 tissue sample;
79 tissue sample;
80 laser beam;
81 laser beam;
82 reflected laser beam;
83 laser beam;
84 laser beam;
85 reflected laser beam;
86 laser beam of sealing;
87 laser beam of cutting;
95 optical fiber;
96 optical fiber;
99 distance between the distal ends of sealing and cutting optical fibers;
150 tissue cut area;
151 tissue coagulation zone (due to tissue sealing laser sealing all small blood vessels);
152 deep penetration zone;
153 tissue;
301 depth sensor via guided optical fibers;
302 pyrometer via guided optical fibers;
1001 laser beam delivery unit;
1002 laser system;
1003 laser system of two laser sources;
5001 smart laser tissue sealing and cutting apparatus;
5002 smart laser tissue sealing and cutting apparatus with two laser sources; and
5003 smart laser tissue sealing and cutting apparatus with two laser sources.

### DETAILED DESCRIPTION OF PREFERRED EMBODIMENTS

### Overview

During surgeries on a human patient or an animal patient, tissue sealing and cutting is intergraded part of surgical operations that a surgical tool such as electrosurgical units are used to perform tissue sealing and cutting functions. With intrinsic of hazard the electrical current based tissue sealing and cutting of electrosurgical units, the smart laser system replaces for tissue sealing and cutting operations. Its use is more prevalent in laparoscopic and robotic surgeries where grounding is difficult to achieve for electrosurgical units. Metal surgical and probe tools of the laparoscopic and robotic surgery exacerbate the risk factor.

During a laparoscopic and robotic surgical operation, one of instrument arms may be fitted with a monopolar instrument of the electrosurgical unit. The functions of monopolar instrument are to seal and cut a tissue of the patient. Another electrosurgical unit called bipolar is commonly used to seal a large vessel.

One way to eliminate the risk from the electrosurgical units is to use non-electrical current based system such as a laser tissue sealing and cutting apparatus described herein. Another non-electrical system commercially available is an ultrasonic based instrument, which is mostly used as a vessel sealer in function similar to the bipolar instrument. During a surgery, the monopolar instrument is a choice of surgical instrument to seal and cut a tissue because electrical grounding is through tissue of an intended area of seal and cut. There are many laser systems being used in the medical field; however, none of them is suitable for use in a laparoscopic and robotic surgery environment to replace the monopolar instrument. Most laser systems do not have a dual function of sealing and cutting. They also do not have a smart sensing system to monitor tissue conditions and prevent thermal damages during tissue seal and cut operations.

A laser system to replace the monopolar instrument in laparoscopic and robotic surgeries requires a dual function of tissue seal and cut. When the tissue is being sealed or cut, the laser system described herein switches to a tissue seal or cut function, respectively. Switching to a seal or cut state is executed mechanically by a motor and a switching plate in the laser system. Another way that it can be done is optically with a motor and a mirror. Another laser system described herein is two laser sources without a switching mechanism. One laser source provides sealing power, and the other provides cutting power. When the tissue is being sealed or cut, one laser source turns on, and the other goes to a standby mode. For a single laser source with a switching mechanism, the laser source consists of diode pumped or Nd:YAG laser. For a dual laser source, diode pumped or Nd:YAG laser is used for sealing, and Nd:YAG, Ho:YAG or Er:YAG laser is used for cutting. All laser sources are in a power range of 20 to 120 watt.

When laser light interacts with human tissue, there may be many issues related to burning of the tissue, and it is difficult to detect by untrained medical doctor or hospital staff. One way to reduce the risk of unwanted tissue burning using any laser interacting with human tissue is to develop a laser sensing system that will self-control the laser interaction process to prevent tissue burning. In order to achieve the self-controlled laser interaction with human tissue, a novel laser sensing system is provided herein.

The combination of the laser pulse modulation of the tissue sealing laser source 20 and photodiode 42 provides a closed-loop feedback system to prevent the emission of any excess laser energy to prevent burning of the tissue. The closed-loop feedback system also prevents under delivery of the laser energy to result in premature termination of tissue sealing or tissue cutting procedure.

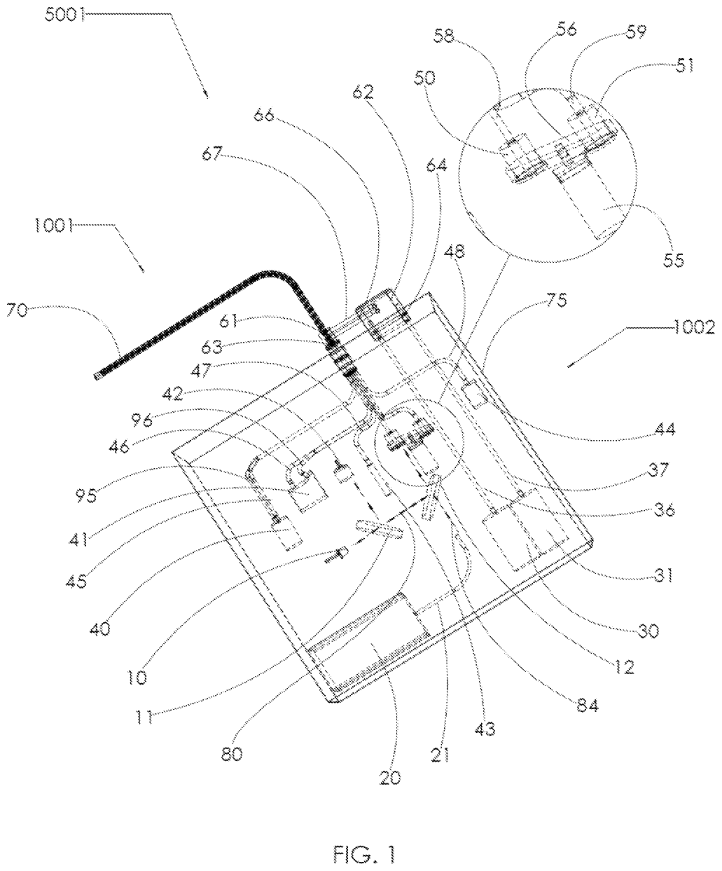

Referring FIG. 1, the laser beam delivery unit 1001 is a single use and disposable. Its purpose is to deliver laser beams to a tissue and send reflected laser beams back to sensor receivers in the laser system. The outer sheath of the laser beam delivery unit holds all fiber optic assemblies together in bundle. The laser beam delivery unit contains at least two optical fibers, one for laser sealing and the other for laser cutting. Optical fiber guided sensors presented herein are a pyrometer (or non-contact temperature sensor), depth sensor, spectral sensor and guide laser. A non-contact temperature sensor such as pyrometer detects tissue temperature via an optical fiber. A depth sensor measures a distance between the probing end of an optical fiber and a tissue. The distance measurement is performed technologies based on the time of flight or triangulation via an optical fiber. A spectral sensor is used to perform multi spectrum analysis to detect and determine tissue and cell types via an optical fiber. A guide laser is used as a laser pointer to where a tissue is targeted for sealing and cutting. It may be also used as a probing laser for the optical feedback sensing technology. In addition, water and air tanks are installed for irrigation. If air and water line infrastructure are available, they will be directed hook up to the infrastructure. A temperature sensor of thermocouple or resistance temperature detector is installed in the laser system.

FIG. 1 illustrates a smart laser tissue sealing and cutting apparatus 5001 in accordance with an embodiment of the present disclosure. In the laser tissue sealing and cutting apparatus 5001, the laser source 20, Nd:YAG or diode pumped laser, is used for operations of sealing and cutting of a tissue. Switching to a seal or cut state is executed mechanically by the motor 55 and the switching plate 56 in the laser system 1002. When the tissue is being cut, the motor 55 rotates and aligns the laser coupling 50 to the laser output fiber 21. The laser beam 84 is delivered to the laser coupling 50 and transmitted via the optical fiber 58 to the distal end for tissue cutting operation. When the tissue is being sealed, the motor 55 rotates and aligns the laser coupling 51 to the laser output fiber 21. The laser beam 84 is delivered to the laser coupling 51 and transmitted via the optical fiber 59 to the distal end for tissue sealing operation. A switching operation shown here is an example of a mechanical switching. The switching can be done optically as well with a motor and a mirror.

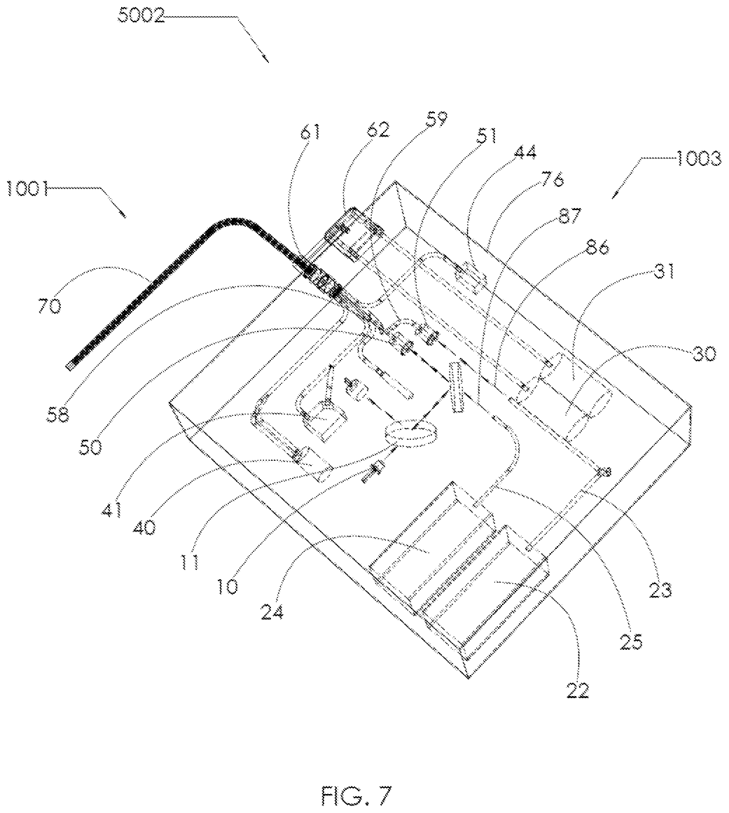

FIG. 7 illustrates another tissue sealing and cutting apparatus 5002 with two laser sources 22 and 24 in accordance with an embodiment of the present disclosure. The laser system 5002 contains two laser sources 22 and 24 and no switching mechanism. One laser source 22 provides sealing power, and the other 24 provides cutting power. When the tissue is being sealed, the laser source 22 turns on and the other 24 goes to a standby mode. Then, the laser beam 86 is delivered to the laser coupling 51 and transmitted via the optical fiber 59 to the distal end for tissue sealing operation. When the tissue is being cut, the laser source 24 turns on and the laser source 22 goes to a standby mode. Then, the laser beam 87 is delivered to the laser coupling 50 and transmitted via the optical fiber 58 to the distal end for tissue cutting operation. The seal and cut operations can be performed simultaneously, turning on both the laser source 22 and 24 at the same time. The laser source 20 uses diode pumped or Nd:YAG laser for tissue sealing and cutting. The laser source 22, which is used for sealing, uses diode pumped or Nd:YAG laser. For the laser source 24, it uses Nd:YAG, Ho:YAG or Er:YAG laser. All laser sources are configured to deliver a high-power laser beam in CW or pulse-mode to deliver 20 to 120 watts of laser power into a given tissue volume.

At the distal end of fiber, the distance 99 defines a height difference between tissue sealing and cutting operations away from a tissue. The tissue cutting is operated at a distance between the tissue level and 100 mm away from the tissue. The tissue sealing is operated at a distance between 5 mm and 200 mm away from a tissue. Laser power and/or laser frequency modulation (CW or pulsed) is provided different for laser tissue cutting and sealing operations. The distal end of the cutting optical fiber 58 is coated with an optical coating such as antireflective, high-reflector, dielectric and/or Teflon to supplant precarbonization of the cutting fiber tip prior to cutting operation.

The photodiode 42 and probe laser 10 may be used for the optical feedback technology for sensing tissue conditions and preventing tissue damages. Optical fiber guided sensors, pyrometer 40, depth sensor 41, spectral sensor and guide laser 43, are presented herein. Fiber optics are used to transmit light energy over long distances. Optical fibers are made of optical quality glass or plastic that are in a diametric range of between 0.1 and 2 mm. In photoelectric sensing, these fibers are used to transmit and/or receive light from an emitter such as a LED or laser diode of a sensor. The pyrometer 40 sends the laser beam 83 from an emitter to a tissue via the optical fiber 45. The reflected beam 85 off from the tissue 78 is transmitted back to a receiver in the pyrometer 40 through the optical fiber 95. A returned signal provides temperature information of tissue. The depth sensor 41 sends the laser beam 81 from an emitter to a tissue via the optical fiber 46. The reflected beam 82 off from the tissue 79 is transmitted back to a receiver in the depth sensor 41 through the optical fiber 96. A returned signal provides distance information of the optical fiber 81 away from the tissue 79. A depth distance off from the tissue 79 is measured by means of a triangulation or time of flight method. The guide laser 43 sends a laser beam from an emitter to a tissue via the optical fiber 47. The guide laser beam is used as a laser pointer to illuminate an area where tissue sealing, cutting and sensing are performed. Its reflected laser beam from the tissue may be used as an optical feedback signal of the photodiode 42. The wavelength of the guide laser 43 is in a visible range of light with a color of green, red or blue. A spectral sensor for multi spectrum analysis works like the pyrometer 40 and depth sensor 41.

Two non-optical fiber probes are presented herein. The temperature sensor 44 measures temperatures of the tissue by thermocouple 48 or resistance temperature detection. The air tank 30 and water tank 31 provide air and water via tubing 36 and 37, respectively. The air tank 30 and water tank 31 may not need if water and air lines are provided from a building infrastructure.

Referring to FIG. 1, the smart tissue sealing and cutting apparatus 5001 utilizes temperatures and data from optical fiber probe sensors and calculates an optimum tissue cutting and sealing time and power level using a CPU in the system. In addition, feedback control data from the photodiode 42 provide energy level information. The smart laser sensing method and laser modulation techniques provide the laser system to achieve safer and effective tissue sealing and cutting for any type of medical procedure.

The use of the smart laser sensing system, e.g., the tissue sealing and cutting apparatus 5001, is to minimize any uncertainty of medical procedure, by using a closed loop feedback system. Many medical devices failed on over-delivery of too much energy by means of RF power, electrical power or ultrasonic energy. In contrast, the tissue sealing and cutting apparatus 5001 is designed with a precision control of delivered laser energy by pulse modulation and smart sensing techniques to overcome most issues encountered by conventional devices and approaches.

The laser tissue sealing and cutting apparatus 5001 includes at least three unique inventive concepts. Firstly, the tissue sealing and cutting apparatus 5001 and 5002 performs both tissue sealing and cutting operations. The single laser source unit 5001 modulates tissue sealing and cutting by the laser source 20 and switch plate 56. The dual laser source unit 5002 operates tissue sealing and cutting from two laser sources 22 and 24, respectively. Both laser sources 22 and 24 can be operated simultaneously.

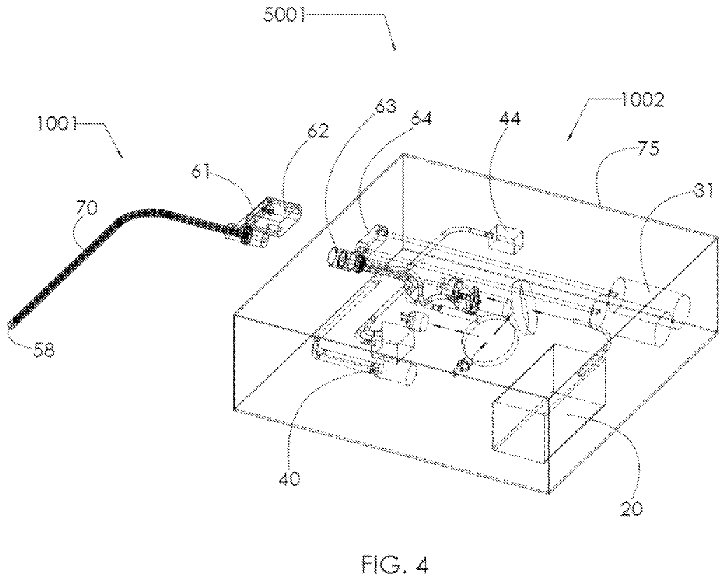

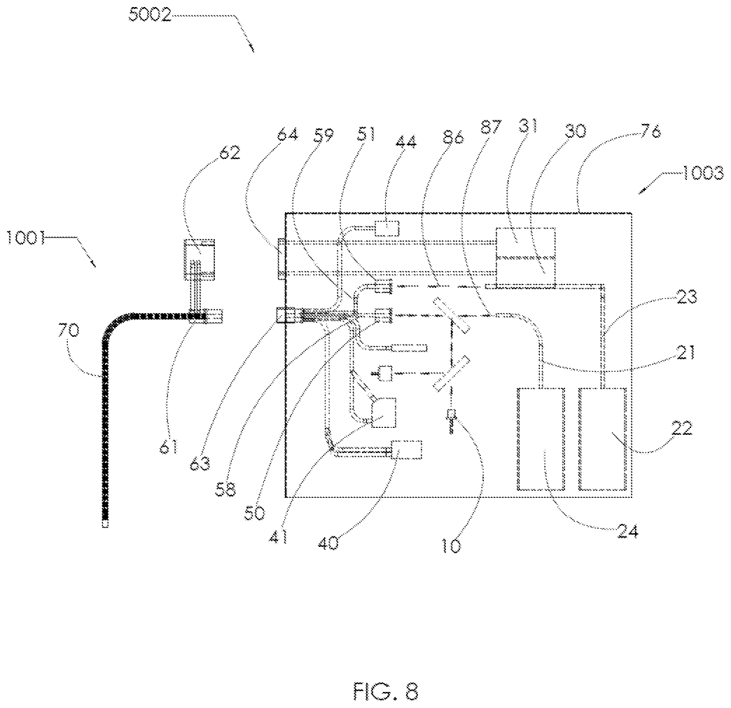

Secondly, the laser beam delivery unit 1001 is a single use and disposable, which is a requirement of most surgical devices. Referring to FIGS. 4 and 8, the laser beam delivery unit 1001 is detached from the laser system 1002 and 1003. The fiber optic coupling is made from the male coupler 61 and the female coupler 63.

Thirdly, the tissue sealing and cutting apparatus 5001 includes optical fiber guided sensors, pyrometer 40, depth sensor 41, spectral sensor and guide laser 43. These sensors are operated normally without use of optical fibers. The use of optical fibers allows sensing to be performed in an inaccessible area. Measurements from all these optical sensors are critical to an overall energy distribution calculation for algorithm development and tissue/cell type detections.

Fourthly, the distal end of the laser cutting optical fiber 58 is coated with an optical coating such as antireflective, high-reflector, dielectric and Teflon. The purpose of the optical coating is to supplant precarbonization of the cutting tip prior to laser cutting operation. Normally, precarbonization of the cutting tip is performed on a sterile cork or wooden spatula prior to cutting operation. The laser beam delivery unit 1001 may be supplied with the standard precarbonization of the sterile cork already performed. The precarbonization only applies to diode pumped and Nd:YAG laser in a laser cutting operation.

Various embodiments disclosed herein pertain to an apparatus and method for sealing and cutting of tissue during general, endoscopic, laparoscopic and robotic surgeries. The apparatus comprises a laser system and a laser beam delivery unit. The laser system emits tunable laser beam with a wavelength suitable for sealing and cutting of a soft tissue. The laser beam delivery unit is disposable and is used to guide the laser beam to where tissue sealing and cutting is needed. The optical feedback and sensing technologies provide a safe and effective energy delivery method to perform tissue sealing and cutting. The smart laser tissue sealing and cutting apparatus 5001 and 5002 could supplant the current monopolar electrosurgical device, which possesses an electric shock hazard.

Therefore, an apparatus in accordance with the present disclosure provides a number of advantages over electrosurgical devices and other existing approaches. The advantageous include, but are not limited to, the following:

(1) a laser system with tissue sealing and cutting capabilities;

(2) a single use and disposable laser beam delivery unit;

(3) an optical feedback sensing technology using a photodiode sensor via an optical fiber;

(4) tissue temperature data acquisition with non-contact temperature sensor such a pyrometer via optical fibers;

(5) depth distance data acquisition with a depth sensor via optical fibers;

(6) multi spectrum analysis using a spectral sensor via optical fibers;

(7) a guide laser functioned as a laser pointer and target illumination via an optical fiber;

(8) the cutting tip precarbonization prior to laser cut operation in a single laser system.

(9) a smart laser system utilizing above sensor data for algorithm development and tissue/cell type detection.

Illustrative Implementations

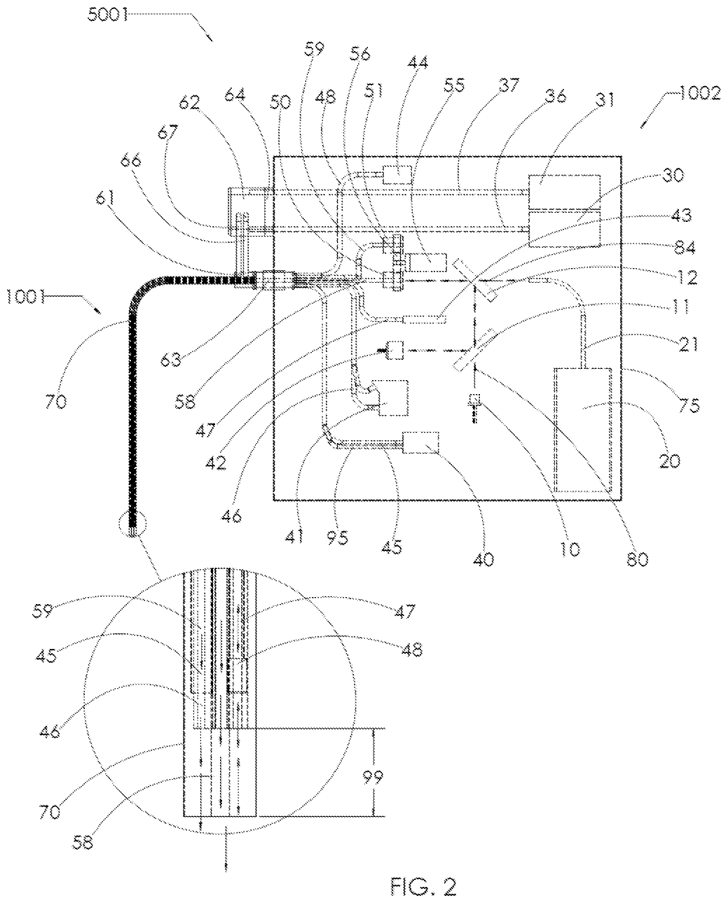

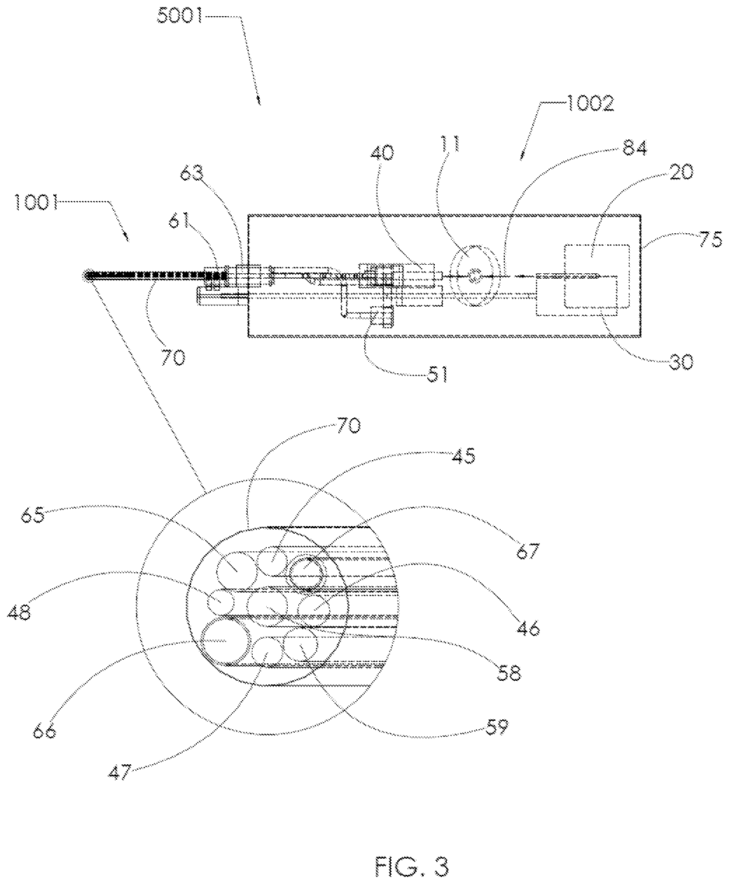

FIG. 1 illustrates a tissue sealing and cutting apparatus 5001 in accordance with an embodiment of the present disclosure. FIG. 2 is a plan view of the tissue sealing and cutting apparatus 5001. FIG. 3 is an enlarged view of a probe tip of the tissue sealing and cutting apparatus 5001. FIG. 4 is a detachment of the laser beam delivery unit from the laser system. The following description refers to FIGS. 1-4.

As shown in FIG. 1, apparatus 5001 may comprise a laser system 1002 and a laser beam delivery unit 1001. The laser system 1002 may comprise the laser source 20 that is configured to emit a laser beam suitable for cutting to the laser coupling 50, which transmits the beam through the optical fiber 58. When tissue is being sealed, the motor 55 rotates the switchable plate 56 to align the laser coupling 51 to the laser output fiber 21 for tissue sealing operation. The laser beam 84 tuned for sealing power and frequency is delivered to the laser coupling 51 and transmitted via the optical fiber 59 to a tissue for sealing operation. When tissue is being cut, the motor 55 rotates the switchable plate 56 to align the laser coupling 50 to the laser output fiber 21 for tissue cut operation. The laser beam 84 tuned for cutting power and frequency is delivered to the laser coupling 50 and transmitted via the optical fiber 58 to a tissue for cutting operation. A switching operation shown here is an example of a mechanical switching. The switching can be done optically as well with a motor and a mirror. Detachment of the laser beam delivery unit 1001 from the laser system 1002 occurs at the mechanical coupling port 61 and 63 for optical fibers and the mechanical coupling block 62 and 64 for water and air lines. The laser beam delivery unit 1001 is a single use such that after each use it may be disposed of while the laser system 1002 is made to be re-used and serviceable.

FIG. 7 illustrates another tissue sealing and cutting apparatus 5002 with two laser sources 22 and 24 in accordance with an embodiment of the present disclosure. FIG. 8 is a plan view of the tissue sealing and cutting apparatus 5002 with the laser beam delivery 1001 detached. The following description refers to FIGS. 7 and 8.

The tissue sealing and cutting apparatus 5002 contains two laser sources 22 and 24 and no switching mechanism. The laser source 22 provides sealing power, and the other laser source 24 provides cutting power. When the tissue is being sealed, the laser source 22 turns on and the other 24 goes to a standby mode. The laser beam 86 is delivered to the laser coupling 51 and transmitted via an optical fiber 59 to a tissue for sealing operation. When the tissue is being cut, the laser source 24 turns on and the laser source 22 goes to a standby mode. The laser beam 87 is delivered to the laser coupling 50 and transmitted via the optical fiber 58 to a tissue for cutting operation. Both laser source 22 and 24 can be operated simultaneously.

In one embodiment, at least one parameter of the laser beam 84 may be tunable for cutting or sealing. For example, a frequency of the laser beam 84, a power level of the laser beam 84, or a combination thereof, may be tunable.

In one embodiment, the laser beam 84 may be tunable to be suitable for sealing or cutting a tissue. For example, the laser unit 20 may be configured such that the laser beam 84 is tunable to a suitable wavelength and/or power level and/or pulsing frequency for the laser beam to seal or cut tissue. In other words, apparatus 5001 may be dual-functional in that it is not only configured to seal tissue but also cut tissue for surgical purposes.

In one embodiment, the laser source 20, 22, 24 may comprise a solid state laser, a fiber laser, or a combination thereof. In other embodiments, the laser unit 20 may comprise Nd:YAG, diode pumped laser or a combination thereof. The laser source 24 comprises Nd:YAG, diode pumped laser or a combination thereof. The laser source 22 comprises Nd:YAG, Er:YAG, Ho:YAG laser or a combination thereof.

In one embodiment, the laser source 20 may emit the laser beam 84 in a pulsed mode.

In one embodiment, the laser beam delivery unit 1001 may comprise at least one cutting 58 and one sealing 59 optical fibers. The outer sheathing 70 contains 3 to 16 optical fibers of glass and plastic.

In one embodiment, the combination of the laser pulse modulation of the photodiode 42 and probing laser 10 provides a closed-loop feedback system to prevent the emission of any excess laser energy to prevent burning of the tissue. The closed-loop feedback system also prevents under delivery of the laser energy to result in premature termination of tissue sealing or cutting procedure. The combination of laser pulse modulation and smart sensing techniques of the present disclosure provides a laser tissue sealing and cutting device, e.g., the apparatus 5001, to minimize interference with medical doctor and staff to thereby improve laser tissue sealing and cutting technique.

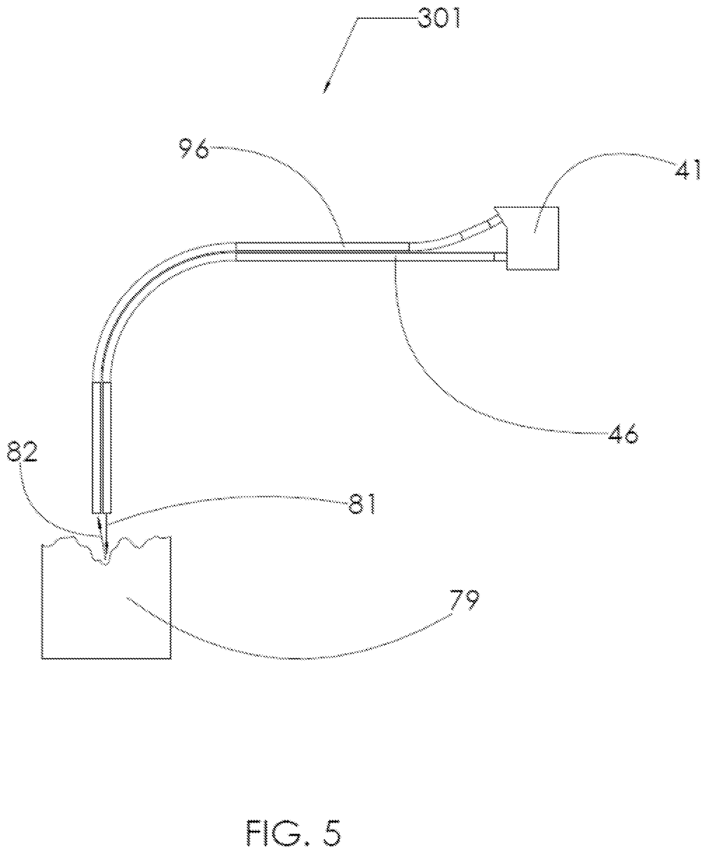

In one embodiment, the laser beam delivery unit 1001 may comprise the depth sensor 41 to measure a distance between the distal end of the optical fiber 46 and the tissue 79. According to FIG. 5, the depth sensor 41 emits the laser beam 81 to the tissue 79 via the optical fiber 46. The reflected laser beam 82 is transmitted through the optical fiber 96 to a receiver in the depth sensor 41. The method of detection is based on the triangulation or time of flight technology.

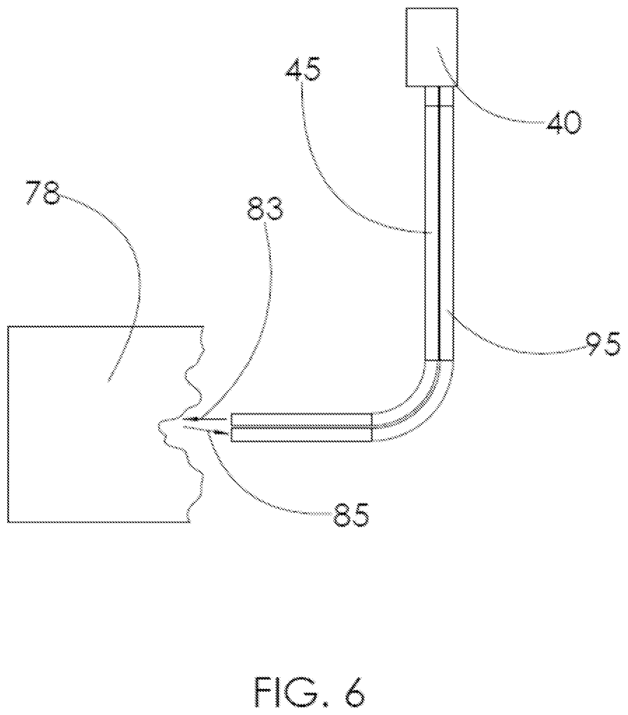

In one embodiment, the laser beam delivery unit 1001 may comprise the pyrometer 40 for remote tissue temperature detection. According to FIG. 6, the pyrometer 40 emits the laser beam 83 to the tissue 78 via the optical fiber 45. The reflected laser beam 85 is transmitted through the optical fiber 95 to a receiver in the pyrometer 40.

In one embodiment, the laser beam delivery unit 1001 may further comprise the guide laser 43 for a laser beam target illumination. This also could be a signal laser beam for the optical feedback technology.

In one embodiment, the laser beam delivery unit 1001 may further comprise the temperature sensor 44 to measure temperatures of the tissue via the temperature sensor wire 48. The temperature detection is based on the thermocouple or resistance temperature detection.

In one embodiment, the laser beam delivery unit 1001 may further comprise one air and one water dispensing tube of 66 and 67, respectively.

In one embodiment, the laser system 1002 may further comprise one air and one water tank of 30 and 31, respectively.

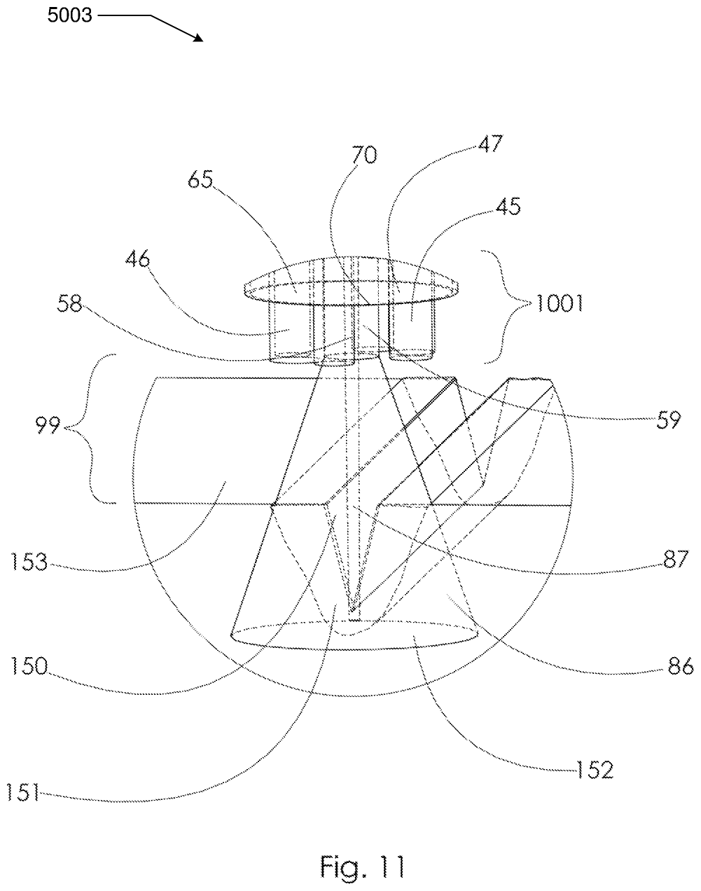

FIG. 11 illustrates yet another tissue sealing and cutting apparatus 5003 with a laser system and a laser beam delivery unit 1001. The laser system may include two lasers (or two laser sources), namely: (a) a tissue cutting laser (not shown) configured to emit a first laser beam 87 to cut a tissue 153, and (b) a tissue sealing laser (not shown) configured to emit a second laser beam 86 to seal the tissue 153. The laser beam delivery unit 1001 may be detachably coupled to the laser system, and the laser beam delivery unit 1001 may be configured to guide and direct the first and second laser beams 87 and 86 to cut and seal the tissue 153. FIG. 11 also shows a tissue cut area 150, a tissue coagulation zone 151 (due to tissue sealing by second laser beam 86 sealing all small blood vessels), and a deep penetration zone 152.

In some cases, the laser beam delivery unit 1001 may include a fiber optic sheathing 70 coupled to the laser system. The fiber optic sheathing 70 may contain a number of optical fibers configured to function as a delivery guide for sensor signals and for the first and second laser beams 87 and 86 to perform tissue cutting and sealing. For instance, fiber optic sheathing 70 may contain a optical fiber 45 for a pyrometer (not shown), an optical fiber 46 for a depth sensor (not shown), an optical fiber 47 for a guide laser (not shown), an optical fiber 58 configured to guide the first laser beam 87 to perform tissue cutting, an optical fiber 59 configured to guide the second laser beam 86 to perform tissue sealing, and an optical fiber 65 for a reflected laser beam to photodiode 42. In some cases, optical fiber 65 may be configured to transmit a reflected laser beam, which is reflected by the tissue 153, from the tissue 153 to a sensor detector of the laser system.

In some cases, optical fiber 58 may have a lower numerical aperture (NA) than optical fiber 59. Alternatively, or additionally, a size of a laser spot on the tissue 153 produced by the second laser beam 86 from optical fiber 59 is equal to or larger than a size of a laser spot on the tissue 153 produced by the first laser beam 87 from optical fiber 58.

In some cases, the laser system may be configured to fire the tissue cutting laser and the tissue sealing laser simultaneously to cut and coagulate the tissue 153. Alternatively, the laser system may be configured to first fire the tissue sealing laser to coagulate the tissue 153 and then fire the tissue cutting laser to cut the tissue 153. Alternatively, the laser system may be configured to first fire the tissue cutting laser to cut the tissue 153 and then fire the tissue sealing laser to coagulate the tissue 153.

In some cases, the tissue cutting laser may be operable in a tissue ablation mode.

In some cases, the laser system may include a pyrometer (not shown) configured to detect a tissue temperature via optical fiber 45 of the laser beam delivery unit 1001. Alternatively, or additionally, the laser system may include a depth sensor (not shown) configured to detect a distance by which a distal end of an optical fiber (e.g., optical fiber 46, optical fiber 58 and/or optical fiber 59) of the laser beam delivery unit 1001 is away from the tissue 153. Alternatively, or additionally, the laser system may include a thermocouple (not shown) or a resistance temperature detector (not shown) configured to detect a temperature of a distal end of optical fiber 58 used in guiding first laser beam 87 to perform tissue cutting or a distal end of optical fiber 59 used in guiding second laser beam 86 to perform tissue sealing. Alternatively, or additionally, the laser system may include a guide laser (not shown) configured to deliver a laser beam via an optical fiber of the laser beam delivery unit 1001 to illuminate a location on or near the tissue 153 where the first and second laser beams 87 and 86 used in tissue cutting and sealing are delivered. For instance, a guide laser beam from the guide laser may be delivered via optical fiber 47.

In some cases, a wavelength of the guide laser may be green color. Accordingly, the green color of the guide laser may be used in monitoring a condition of the tissue 153 after cauterizing by the second laser beam 86 from the tissue sealing laser. Moreover, the green color of the guide laser may be used in monitoring a condition of the tissue 153 after cutting by the first laser beam 87 from the tissue cutting laser.

In some cases, a wavelength of the guide laser may be blue color. Accordingly, the blue color of the guide laser may be used in monitoring a condition of the tissue 153 after cauterizing by the second laser beam 86 from the tissue sealing laser. Moreover, the blue color of the guide laser may be used in monitoring a condition of the tissue 153 after cutting by the first laser beam 87 from the tissue cutting laser.

Advantageously, it is believed that apparatus 5003 may provide an optimum process of laser sealing or cutting of tissue to minimize bleeding and prevent tissue damage. Apparatus 5003 may be considered a smart surgical laser device that can operate in four modes: seal followed by cut, seal only, cut followed by seal, and seal & cut simultaneous mode. The second laser beam 86 from the sealing laser is designed to coagulate tissue by sealing all small blood vessels, while the first laser beam 87 from the cutting laser is designed to cut tissue 153. In operation, in case a surgeon intends to remove a small piece of organ tissue, sealing and cutting tissue operations can be performed almost simultaneously. The benefits of operating in the seal & cut simultaneous mode are to minimize bleeding while tissue cut; this may prevent tissue burns and reduce seal/cut operation time. Based on these sensor data of sensors measuring light reflection, distance proximity and surface temperature, along with laser parameters, the following information may be obtained: a thermal volume, tissue conditions, laser intensity, surface temperatures and surface bleeding conditions. The information may be processed by an onboard computer, which has an algorithm to perform safe tissue seal/cut operations for different tissue types.

It is noteworthy that, under various proposed schemes in accordance with the present disclosure, tissue sealing and cutting process may be dependent on the energy delivered to an area on the tissue. The tissue sealing laser may be exposed to a large area such as 1 mm² to 10 mm² and the associated pulse energy may deliver 1˜100 joule. A testing performed by a prototype of apparatus 5003 shows 0.5 J/mm² in a 980 nm tissue sealing laser can coagulate muscle tissue in 0.5˜1 second. However, the tissue cutting laser typically requires a very tight beam such as less than 1 mm² area with higher laser power. The duration of the tissue cutting laser in activation or being fired can be longer to cut a large area of tissue. For example, if a size of 5 mm-diameter area is cut by the tissue cutting laser, the tissue sealing laser of 0.8 J/mm² of energy may be delivered to coagulate a 10 mm-diameter area, then the tissue cutting laser may be turned on for 5˜10 seconds to cut the 5 mm-diameter area. In this case, the tissue sealing laser can be fired first for 1 second, then the tissue cutting laser can cut out a 5 mm-diameter tissue. On the other hand, the tissue sealing laser and tissue cutting laser may be simultaneously fired to perform sealing and cutting operations at the same time. Consequently, the tissue sealing laser may be fired again to seal the tissue cut area. The pulse duration of tissue cutting laser and tissue sealing laser may be determined by the energy delivered to cut and seal various types of tissue. Unlike a muscle tissue, an organ tissue such as liver or kidney tissue can bleed easily and, in those cases, the tissue sealing laser may be activated or fired frequently to stop the bleeding with the duration of firing being a few seconds to minutes in a reasonable laser intensity. One of the most distinctive differences between the tissue sealing laser beam and tissue cutting laser beam is that the tissue cutting laser beam has a tight beam spot less than 1 mm² and the beam spot of the tissue sealing laser beam needs to be larger than that of the tissue cutting laser beam. In some implementations, it would be advantageous to dynamically change the beam spot size of the tissue sealing laser between different sizes (e.g., between 1 mm² and 10 mm²) instantly to suit the needs of a surgical operation.

Illustrative Operations

FIG. 2 shows laser beams at the probe tip of the tissue sealing and cutting apparatus 5001. Optical fibers are used to transmit laser signal beams to tissue and reflected laser beams back to detectors in the laser system 1002.

As shown in FIG. 5, the laser signal beam 81 and reflected laser beam 82 of the depth sensor 41. The reflected laser beam 82, which is the laser signal beam 81 reflected off from the tissue 79, returns to a receiver in the depth sensor 41 via the optical fiber 96.

As shown in FIG. 6, the laser signal beam 83 and reflected laser beam 84 of the pyrometer 40. The reflected laser beam 84, which is the laser signal beam 83 reflected off from the tissue 78, returns to a receiver in the pyrometer 40 via the optical fiber 95.

Illustrative Processes

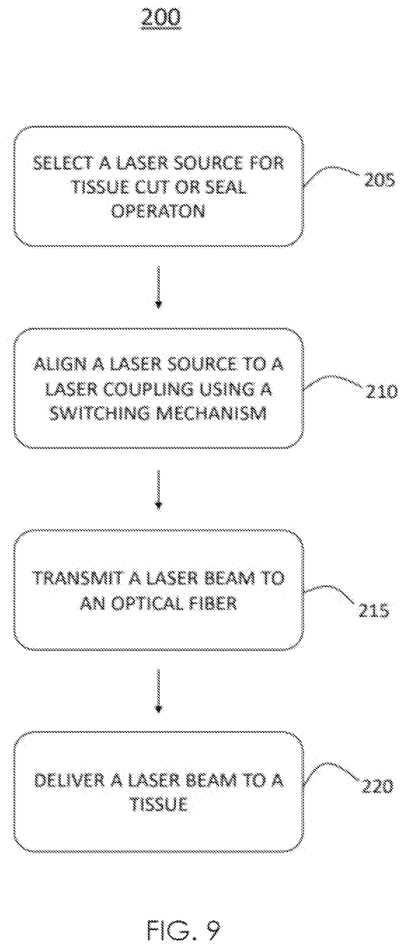

FIG. 9 illustrates a process 200 of sealing and cutting a tissue with an apparatus in accordance with the present disclosure.

Example process 200 includes one or more operations, actions, or functions as illustrated by one or more of blocks 205, 210, 215 and 220. Although illustrated as discrete blocks, various blocks may be divided into additional blocks, combined into fewer blocks, or eliminated, depending on the desired implementation. Further, process 200 may be implemented using apparatus 5001. For illustrative purposes, the operations described below are performed by medical personnel using apparatus 5001. Process 200 may begin at block 205.

At 205, process 200 may comprise selecting a laser source for tissue seal or cut. For example, the laser source 20 may be configured for tissue sealing or cutting.

At 210, process 200 may comprise aligning a laser source to a laser coupling via a switchable mechanism. For example, the alignment of the fiber coupling 50 or 51 to the laser beam 84 of the laser source 20 is done by the switchable plate 56 and the motor 55.

At 215, process 200 may comprise delivering a laser beam to an optical fiber. For example, the laser beam 84 may be transmitted to a tissue via the optical fiber 58 or 59.

At 220, process 200 may comprise delivering a laser beam to a tissue for a sealing or cutting operation. For example, the laser beam 84 is delivered onto a tissue.

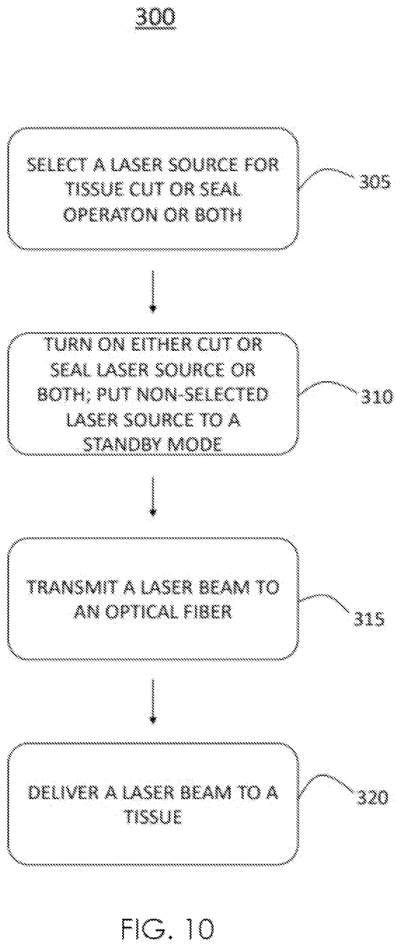

FIG. 10 illustrates a process 300 of sealing and cutting a tissue with an apparatus in accordance with the present disclosure.

Example process 300 includes one or more operations, actions, or functions as illustrated by one or more of blocks 305, 310, 315 and 320. Although illustrated as discrete blocks, various blocks may be divided into additional blocks, combined into fewer blocks, or eliminated, depending on the desired implementation. Further, process 300 may be implemented using apparatus 5002. For illustrative purposes, the operations described below are performed by medical personnel using apparatus 5002. Process 300 may begin at block 305.

At 305, process 300 may comprise selecting a laser source for tissue sealing, cutting or both. For example, the laser source 22 or 24 may be selected for tissue sealing and cutting, respectively. Or both laser source 22 and 24 may be selected for tissue sealing and cutting operations simultaneously.

At 310, process 300 may comprise turning on of either laser cut, seal or both sources. One that isn't selected goes to a standby mode. For example, the laser source 24 may turn on for tissue cutting and the laser source 22 goes to a standby mode. Or both laser source 22 and 24 may be selected for tissue sealing and cutting operations simultaneously, and no laser goes to a standby mode.

At 315, process 300 may comprise delivering a laser beam to an optical fiber. For example, the laser beam 87 may be transmitted to a tissue via the optical fiber 58 for tissue cutting.

At 320, process 300 may comprise delivering a laser beam to a tissue for sealing, cutting and both operations. For example, the laser beam 87 is delivered onto a tissue for cutting.

In one embodiment, process 300 may have both laser sources 22 and 24 turn on for tissue sealing and cutting simultaneously.

Feature Highlights

In view of the above, select features of various embodiments of the present disclosure are highlighted below.

In one aspect, an apparatus may include a laser system and a laser beam delivery unit detachably coupled to the laser system. The laser system may include a tissue cutting laser configured to emit a first laser beam to cut a tissue. The laser system may also include a tissue sealing laser configured to emit a second laser beam to seal the tissue. The laser beam delivery unit may be configured to guide and direct the first and second laser beams to cut and seal the tissue.

In some implementations, the laser beam delivery unit may include a fiber optic sheathing coupled to the laser system. The fiber optic sheathing may contain a number of optical fibers configured to function as a delivery guide for sensor signals and for the first and second laser beams to perform tissue cutting and sealing. In some implementations, one of the number of optical fibers of the fiber optic sheathing may be configured to transmit a reflected laser beam, which is reflected by the tissue, from the tissue to a sensor detector of the laser system.

In some implementations, the laser beam delivery unit may further include (a) a first optical fiber configured to guide the first laser beam to perform tissue cutting; and (b) a second optical fiber configured to guide the second laser beam to perform tissue sealing.

In some implementations, the first optical fiber may have a lower numerical aperture (NA) than the second optical fiber.

In some implementations, a size of a laser spot on the tissue produced by the second laser beam from the second optical fiber may be equal to or larger than a size of a laser spot on the tissue produced by the first laser beam from the first optical fiber.

In some implementations, the laser system may be configured to fire or otherwise activate the tissue cutting laser and the tissue sealing laser simultaneously to cut and coagulate the tissue.

In some implementations, the laser system may be configured to first fire or otherwise activate the tissue sealing laser to coagulate the tissue and then fire or otherwise activate the tissue cutting laser to cut the tissue.

In some implementations, the laser system may be configured to first fire or otherwise activate the tissue cutting laser to cut the tissue and then fire or otherwise activate the tissue sealing laser to coagulate the tissue.

In some implementations, the tissue cutting laser may be operable in a tissue ablation mode.

In some implementations, the laser system may include a pyrometer configured to detect a tissue temperature via one or more optical fibers of the laser beam delivery unit.

In some implementations, the laser system may include a depth sensor configured to detect a distance by which a distal end of an optical fiber of the laser beam delivery unit is away from the tissue.

In some implementations, the laser system may include a thermocouple or a resistance temperature detector configured to detect a temperature of a distal end of a first optical fiber used in tissue cutting or a distal end of a second optical fiber used in tissue sealing.

In some implementations, the laser system may include a guide laser configured to deliver a laser beam via an optical fiber of the laser beam delivery unit to illuminate a location on or near the tissue where the first and second laser beams used in tissue cutting and sealing are delivered.

In some implementations, a wavelength of the guide laser may be green color. In some implementations, the green color of the guide laser may be used in monitoring a condition of the tissue after cauterizing by the tissue sealing laser. Alternatively, or additionally, the green color of the guide laser may be used in monitoring a condition of the tissue after cutting by the tissue cutting laser.

In some implementations, a wavelength of the guide laser may be blue color. In some implementations, the blue color of the guide laser may be used in monitoring a condition of the tissue after cauterizing by the tissue sealing laser. Alternatively, or additionally, the blue color of the guide laser may be used in monitoring a condition of the tissue after cutting by the tissue cutting laser.

In one aspect, an apparatus may include a laser system and a laser beam delivery unit detachably coupled to the laser system. The laser system may include a tissue cutting laser configured to emit a first laser beam to cut a tissue. The laser system may also include a tissue sealing laser configured to emit a second laser beam to seal the tissue. The laser beam delivery unit may be configured to guide and direct the first and second laser beams to cut and seal the tissue. The laser system may further include one or more of the following: (a) a pyrometer configured to detect a tissue temperature via one or more optical fibers of the laser beam delivery unit, (b) a depth sensor configured to detect a distance by which a distal end of an optical fiber of the laser beam delivery unit is away from the tissue, (c) a thermocouple or a resistance temperature detector configured to detect a temperature of a distal end of a first optical fiber used in tissue cutting or a distal end of a second optical fiber used in tissue sealing, and (d) a guide laser configured to deliver a laser beam via an optical fiber of the laser beam delivery unit to illuminate a location on or near the tissue where the first and second laser beams used in tissue cutting and sealing are delivered, with a wavelength of the guide laser being green color or blue color.

Additional and Alternative Implementation Notes

The above-described embodiments and techniques pertain to an apparatus and method for sealing and cutting of tissue during surgeries, especially in laparoscopic and robotic. Although the techniques have been described in language specific to certain applications, it is to be understood that the appended claims are not necessarily limited to the specific features or applications described herein. Rather, the specific features and applications are disclosed as example forms of implementing such techniques.

In the above description of example implementations, for purposes of explanation, specific numbers, materials configurations, and other details are set forth in order to better explain the invention, as claimed. However, it will be apparent to one skilled in the art that the claimed invention may be practiced using different details than the example ones described herein. In other instances, well-known features are omitted or simplified to clarify the description of the example implementations.

The described embodiments are intended to be primarily examples. The described embodiments are not meant to limit the scope of the appended claims. Rather, the claimed invention might also be embodied and implemented in other ways, in conjunction with other present or future technologies.

Moreover, the word “example” is used herein to mean serving as an example, instance, or illustration. Any aspect or design described herein as “example” is not necessarily to be construed as preferred or advantageous over other aspects or designs. Rather, use of the word example is intended to present concepts and techniques in a concrete fashion. The term “techniques,” for instance, may refer to one or more devices, apparatuses, systems, methods, articles of manufacture, and/or computer-readable instructions as indicated by the context described herein.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. An apparatus, comprising:
a laser system comprising:
a tissue cutting laser configured to emit a first laser beam to cut a tissue, and
a tissue sealing laser configured to emit a second laser beam to seal the tissue; and
a laser beam delivery unit detachably coupled to the laser system, the laser beam delivery unit configured to guide and direct the first and second laser beams to cut and seal the tissue.

2. The apparatus of claim 1, wherein the laser beam delivery unit comprises:
a fiber optic sheathing coupled to the laser system, the fiber optic sheathing containing a number of optical fibers configured to function as a delivery guide for sensor signals and for the first and second laser beams to perform tissue cutting and sealing,
wherein one of the number of optical fibers of the fiber optic sheathing is configured to transmit a reflected laser beam, which is reflected by the tissue, from the tissue to a sensor detector of the laser system.

3. The apparatus of claim 2, wherein the laser beam delivery unit further comprises:
a first optical fiber configured to guide the first laser beam to perform tissue cutting; and
a second optical fiber configured to guide the second laser beam to perform tissue sealing.

4. The apparatus of claim 3, wherein the first optical fiber has a lower numerical aperture (NA) than the second optical fiber.

5. The apparatus of claim 4, wherein a size of a laser spot on the tissue produced by the second laser beam from the second optical fiber is equal to or larger than a size of a laser spot on the tissue produced by the first laser beam from the first optical fiber.

6. The apparatus of claim 1, wherein the laser system is configured to fire the tissue cutting laser and the tissue sealing laser simultaneously to cut and coagulate the tissue.

7. The apparatus of claim 1, wherein the laser system is configured to first fire the tissue sealing laser to coagulate the tissue and then fire the tissue cutting laser to cut the tissue.

8. The apparatus of claim 1, wherein the laser system is configured to first fire the tissue cutting laser to cut the tissue and then fire the tissue sealing laser to coagulate the tissue.

9. The apparatus of claim 1, wherein the tissue cutting laser is operable in a tissue ablation mode.

10. The apparatus of claim 1, wherein the laser system comprises a pyrometer configured to detect a tissue temperature via one or more optical fibers of the laser beam delivery unit.

11. The apparatus of claim 1, wherein the laser system comprises a depth sensor configured to detect a distance by which a distal end of an optical fiber of the laser beam delivery unit is away from the tissue.

12. The apparatus of claim 1, wherein the laser system comprises a thermocouple or a resistance temperature detector configured to detect a temperature of a distal end of a first optical fiber used in tissue cutting or a distal end of a second optical fiber used in tissue sealing.

13. The apparatus of claim 1, wherein the laser system comprises a guide laser configured to deliver a laser beam via an optical fiber of the laser beam delivery unit to illuminate a location on or near the tissue where the first and second laser beams used in tissue cutting and sealing are delivered.

14. The apparatus of claim 13, wherein a wavelength of the guide laser is green color.

15. The apparatus of claim 14, wherein the green color of the guide laser is used in monitoring a condition of the tissue after cauterizing by the tissue sealing laser.

16. The apparatus of claim 14, wherein the green color of the guide laser is used in monitoring a condition of the tissue after cutting by the tissue cutting laser.

17. The apparatus of claim 13, wherein a wavelength of the guide laser is blue color.

18. The apparatus of claim 14, wherein the blue color of the guide laser is used in monitoring a condition of the tissue after cauterizing by the tissue sealing laser.

19. The apparatus of claim 14, wherein the blue color of the guide laser is used in monitoring a condition of the tissue after cutting by the tissue cutting laser.

20. An apparatus, comprising:
a laser system comprising:
a tissue cutting laser configured to emit a first laser beam to cut a tissue, and
a tissue sealing laser configured to emit a second laser beam to seal the tissue; and
a laser beam delivery unit detachably coupled to the laser system, the laser beam delivery unit configured to guide and direct the first and second laser beams to cut and seal the tissue,
wherein the laser system further comprises one or more of:
a pyrometer configured to detect a tissue temperature via one or more optical fibers of the laser beam delivery unit,
a depth sensor configured to detect a distance by which a distal end of an optical fiber of the laser beam delivery unit is away from the tissue,
a thermocouple or a resistance temperature detector configured to detect a temperature of a distal end of a first optical fiber used in tissue cutting or a distal end of a second optical fiber used in tissue sealing, and
a guide laser configured to deliver a laser beam via an optical fiber of the laser beam delivery unit to illuminate a location on or near the tissue where the first and second laser beams used in tissue cutting and sealing are delivered,
wherein a wavelength of the guide laser is green color or blue color.
